(19) United States
(12) Patent Application Publication
(10) Pub. No.: US 2020/0249152 A1
(43) Pub. Date: Aug. 6, 2020

(54) SYSTEM OF MEASURING CONTAMINATION OF A WATER SAMPLE

(71) Applicants: George Alexandru Botos, Oakville (CA); Aditya Mardia, Toronto (CA)

(72) Inventors: George Alexandru Botos, Oakville (CA); Aditya Mardia, Toronto (CA)

(21) Appl. No.: 16/782,014

(22) Filed: Feb. 4, 2020

Related U.S. Application Data
(60) Provisional application No. 62/801,275, filed on Feb. 5, 2019.

(51) Int. Cl. G01N 21/31; G01N 1/38; G01N 33/18

(52) U.S. Cl. CPC G01N 21/314; G01N 2021/3181; G01N 33/1826; G01N 1/38

(57) ABSTRACT

Disclosed herein is a system of measuring contamination of a water sample. The system may include a mixture container comprising a mixture chamber, a sample opening, and a reagent opening. Further, the system may include a light emitting device disposed proximal to the mixing chamber. Further, the system may include a light sensing device disposed proximal to the mixing chamber. Further, the system may include a processing device communicatively coupled with each of the light emitting device and the light sensing device. Further, the processing device may be configured for analyzing the at least one predetermined attribute and the at least one attribute. Further, the system may include a storage device configured for storing the notification. Further, the system may include a presentation device communicatively coupled with the processing device. Further, the presentation device is configured for presenting the notification.

### RELATED APPLICATION(S)

Under provisions of 35 U.S.C. § 119e, the Applicant(s) claim the benefit of U.S. provisional application No. 62/801,275, titled “WATER CONTAMINATION MEASURING SYSTEM”, filed on Feb. 5, 2019 which is incorporated herein by reference.

### TECHNICAL FIELD

The present disclosure relates to the field of measuring and testing. More specifically, the present disclosure relates to a system of measuring contamination of a water sample.

### BACKGROUND

According to the World Health Organization (WHO) 2018 Fact Sheets, in 2015, 71% of the global population (5.2 billion people) used a safely managed drinking-water service—that is, one located on-premises, available when needed, and free from contamination. The remaining 2.1 billion people without safely managed services in 2015, which means they use a drinking water source contaminated. Contaminated water can transmit diseases such as diarrhea, cholera, dysentery, typhoid, and polio. Contaminated drinking water is estimated to cause 502,000 diarrheal deaths each year.

Water quality management is mandatory and strictly regulated, not only for drinking water but also for most of the industries which directly (food and beverage industry) or indirectly (pharmaceutical, health care etc.) use water in their products and services. In the current information and social connectivity era, when consumers can easily access health warnings and recall reports online, companies are wary of losing brand equity and of exposure to lawsuits. Manufacturers in these industries need a proven, rapid, real-time microbiological testing process. Most of these industries use testing methods which take days to weeks to provide results. This delay can result in a high cost for manufacturers in terms of production delays, lost sales, product recalls, litigation costs and damage to brand equity.

Detection of microbes is a challenge for water treatment professionals and limits their capability to measure the effectiveness of various water treatment technologies and adjust the treatments in a reasonable time. As a result, the effectiveness of biocidal treatments in water is currently done mostly indirectly—by measuring the levels of chemical used for treatment and assuming the effectiveness of the treatment correlates with results obtained in laboratory conditions. The results are then verified by sending samples to a microbiology laboratory for microbial testing.

Microbial testing involves processing the water sample and, in most cases, attempting to grow the microorganisms into colonies on a nutrient media, and count the visible colonies to estimate the level of microbial contamination. This method however grossly underestimates the microbial levels, as in most cases the microorganism that survives the biocidal treatment would be injured and will not form visible colonies which will grow slower and will be harder to observe.

According to the US Environmental Protection Agency (EPA), there is an immediate need for better, faster, cheaper, real-time, water contamination monitoring techniques applicable to water security needs.

Therefore, there is a need for improved systems for measuring contamination of a water sample that may overcome one or more of the above-mentioned problems and/or limitations.

### BRIEF SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein system of measuring contamination of a water sample. Further, the system may include a mixture container comprising a mixture chamber, a sample opening, and a nano reagent opening. Further, each of the sample opening and the nano reagent opening lead in to the mixing chamber. Further, the mixture chamber may be configured for storing a mixture of the water sample and a nano reagent. Further, the water sample may include at least one contaminant. Further, the nano reagent may include a plurality of functionalized nanoparticles. Further, the system may include a light emitting device disposed proximal to the mixing chamber. Further, the light emitting device may be configured for emitting an incident light wave corresponding to at least one predetermined attribute. Further, the incident light wave may be directed to pass through the mixture. Further, the system may include a light sensing device disposed proximal to the mixing chamber. Further, the light sensing device may be configured for receiving a transmitted light wave. Further, the transmitted light wave may be associated with the incident light wave. Further, the transmitted light wave may be associated with at least one attribute. Further, the system may include a processing device communicatively coupled with each of the light emitting device and the light sensing device. Further, the processing device may be configured for analyzing the at least one predetermined attribute and the at least one attribute. Further, the processing device may be configured for generating a notification based on the analyzing. Further, the system may include a storage device configured for storing the notification. Further, the system may include a presentation device communicatively coupled with the processing device. Further, the presentation device may be configured for presenting the notification.

Further disclosed herein is system of measuring contamination of a water sample. Further, the system may include a mixture container comprising a mixture chamber, a sample opening, and a nano reagent opening. Further, each of the sample opening and the nano reagent opening lead into the mixing chamber. Further, the mixture chamber may be configured for storing a mixture of the water sample and a nano reagent. Further, the water sample may include at least one contaminant. Further, the nano reagent may include a plurality of functionalized nanoparticles. Further, the system may include a light emitting device disposed proximal to the mixing chamber. Further, the light emitting device may be configured for emitting an incident light wave corresponding to at least one predetermined attribute. Further, the incident light wave may be directed to pass through the mixture. Further, the system may include a light sensing device disposed proximal to the mixing chamber. Further, the light sensing device may be configured for receiving a transmitted light wave. Further, the transmitted light wave may be associated with the incident light wave. Further, the transmitted light wave may be associated with at least one attribute. Further, the system may include a processing device communicatively coupled with each of the light emitting device and the light sensing device. Further, the processing device may be configured for analyzing the at least one predetermined attribute and the at least one attribute. Further, the processing device may be configured for generating a notification based on the analyzing. Further, the system may include a storage device configured for storing the notification. Further, the system may include a presentation device communicatively coupled with the processing device. Further, the presentation device may be configured for presenting the notification. Further, the system may include a communication device communicatively coupled with the processing device. Further, the communication device may be configured for transmitting the notification to at least one user device associated with a user.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system of measuring contamination of a water sample, in accordance with some embodiments.

FIG. 3 is a block diagram of a system of measuring contamination of a water sample, in accordance with some embodiments.

FIG. 4 is a top view of a measuring device configured for measuring contamination of a water sample, in accordance with some embodiments.

FIG. 5 shows a top right-side perspective view of the measuring device.

FIG. 6 is a partial top left-side perspective view of internal structure of the measuring device, in accordance with some embodiments.

FIG. 7 is a schematic showing communication between a measuring unit, a user mobile device, a user computer and a cloud server, in accordance with some embodiments.

FIG. 8 is a snapshot of a user interface of a smartphone application installed on a smartphone, in accordance with some embodiments.

FIG. 9 is a snapshot of a user interface of a smartphone application installed on a smartphone, in accordance with some embodiments.

FIG. 10 is a snapshot of a user interface of a smartphone application installed on a smartphone, in accordance with some embodiments.

FIG. 11 is a flowchart of a method of operating a measuring unit, in accordance with some embodiments.

FIG. 12 is a flowchart of a method of operating a measuring unit, in accordance with some embodiments.

FIG. 13 is a continuation flowchart of the method shown in FIG. 12.

FIG. 14 is a flowchart of a method of operating a measuring unit, in accordance with some embodiments.

FIG. 15 is a continuation flowchart of the method shown in FIG. 14.

FIG. 16 is a continuation flowchart of the method shown in FIG. 14.

FIG. 17 is a schematic showing a filter tip screwed onto syringe used to prepare a water sample, in accordance with some embodiments.

FIG. 18 is a schematic illustrating communication between a cloud server and a computing device, in accordance with some embodiments.

FIG. 19 is a schematic illustrating communication between a measuring unit and a computing unit, in accordance with some embodiments.

FIG. 20 is a block diagram of a computing device for implementing the methods disclosed herein, in accordance with some embodiments.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems of measuring contamination of a water sample, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice-based interface, gesture-based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third-party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role-based access control, and so on.

Further, one or more steps of the method disclosed herein may be initiated, maintained, controlled and/or terminated based on a control input received from one or more devices operated by one or more users such as, for example, but not limited to, an end-user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human-readable secret data (e.g. username, password, passphrase, PIN, secret question, secret answer etc.) and/or possession of a machine-readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human-readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure may describe systems include a measuring device that is pre-calibrated to the detection range specific to the nano reagent used for testing. Further, the system includes the test vials that contain the functionalized nanoparticles included in testing vials for microbial, or chemical reagents for chemical detection. The functionalized nanoparticles are specific to the reagent to be detected. For example, for certain microbial detection, the reagent contains functionalized nanoparticles that have a wavelength shift detectable when mixed with prepared contaminated water samples between 450 to 550 nm and 600 to 700 nm. Further, a proprietary Nano test reagent may be used. Further, the system includes hollow fiber cartridges used to concentrate microbial samples and transfer them into the test vial quantitatively. Further, the system includes software that can read the data from the device sensors/hardware on the measuring device. Further, the system includes software on a mobile phone or a desktop computer that can consume data from the firmware of the measuring device. Further, the system includes software on a mobile phone or a server (such as a cloud server) that can convert the sensor data into a response by selecting the raw data, choosing the relevant equation, generates a slope and estimated contamination level Further, the system includes software on a mobile phone or a desktop that can display this output in a useable format for user. Further, proprietary algorithms may be used to obtain data, generate data, process data and compute results.

Further, the disclosed system allows for performing tests in the field, lab or at home using wireless the device, vials and a smartphone. Further, the disclosed system enables users to find out what's in the water sample within minutes, allowing safe decisions to be made quickly. Further, the disclosed system pushes the result in the cloud to find patterns and enable early warning. Further, the disclosed system may detect the contamination of the sample in a time ranging from 30 seconds to 10 minutes. Further, the disclosed system may detect the contamination level in detection units (or limits). Further, the detection units may include Colony-Forming Unit (CFU), PPM range etc.

According to some embodiments, a water contamination measuring system configuration is disclosed. The water contamination measuring system may include a sampling unit, a reagent, a measuring unit, and a computing unit. Further, the sampling unit may consist of a volumetric sample collection device, which may be a syringe, and a sample concentration cartridge, which for microbial testing can be a filter cartridge that may allow for quantitative transfer of microorganism cells into a reagent vial.

Further, the reagent may consist of a functionalized nanoparticle suspension, which may interact with at least one contaminant in the water samples. For total microbial testing, the reagent used may comprise a suspension of functionalized nanoparticles of customized sizes between 10 nm and 200 nm, designed so that the reagent may attach to the microbial cell membrane in a quantitative manner (for a given microbial species the number of nanoparticles attaching to the surface is consistent).

Further, the measuring unit may collect data on changes of wavelength absorption/transmission at specific wavelengths and send the data from the measuring unit to the computing unit.

Further, the computing unit may include computing devices, such as smartphones, tablets, or laptops, or cloud servers, such as the central server 102, and so on. Further, the computing unit may receive the data transmitted by the measuring unit and analyze the data to determine a most probable value of a contaminant in the water sample and a level of confidence in the result produced. Further, a software algorithm associated with the computing unit is disclosed. Further, the software algorithm may be configured for processing input provided by the measuring unit. Further, the software algorithm may be associated with a set of codes that may be executed by the computing unit. Further, executing the set of codes may facilitate measurement of the contaminant in the water sample. Further, the software algorithm facilitates analyzing of the data received from the measuring unit to generate a score.

The disclosure describes a portable, robust solution (water contamination measuring system) for detecting microbial levels in water samples in minutes, which may be performed anywhere and by anyone with minimal training to solve existing problems. Therefore, a water contamination measuring system may not require specific laboratory and specialized personnel and may detect microbial count and other contaminant levels (heavy metals, chlorine, etc.) in water samples using functionalized nanoparticles. The water contamination measuring system may also be used to detect specific strains of bacteria and output an estimate of chemicals in water such as free chlorine, hydrogen peroxide and lead.

The exemplary embodiments described herein provide a water contamination measuring system using a light measuring device and calculation technologies. Various configurations of the water contamination measuring system may be produced to suit various purposes.

According to some embodiments, the water contamination measuring system may include a sampling unit, a reagent, a measuring unit, and a computing unit. Further, the sampling unit may consist of a volumetric sample collection device, which may be a syringe, and a sample concentration cartridge, which for microbial testing can be a filter cartridge that may allow for quantitative transfer of microorganism cells into a reagent vial.

The reagent may consist of a functionalized nanoparticle suspension, which may interact with at least one contaminant in the water samples. For total microbial testing, the reagent used may comprise a suspension of functionalized nanoparticles of customized sizes between 10 nm and 100 nm, designed so that the reagent may attach to the microbial cell membrane in a quantitative manner (for a given microbial species the number of nanoparticles attaching to the surface is consistent).

Further, the measuring unit may collect data on changes of wavelength absorption/transmission at specific wavelengths and send the data from the measuring unit to the computing unit.

Further, the computing unit may include computing devices, such as smartphones, tablets, or laptops, or cloud servers, such as the central server 102, and so on. Further, the computing unit may receive the data transmitted by the measuring unit and analyze the data to determine a most probable value of a contaminant in the water sample and a level of confidence in the result produced. Results from the computing unit may be saved on the measuring device, computing device, and shown to a user through one or more display devices, such as display devices connected to the computing device. Further, the saved results may be used to monitor and predict contamination levels in the water sources tested.

Further, the measuring unit may include a light measuring device. The light measuring device may include a spectrometer or a colorimeter to measure absorption or reflection of light in a sample.

Further, in an embodiment, the measuring unit may comprise a handheld wireless or wired device/spectrometer that may house a chamber allowing for measurement of the reaction with minimal interference. This chamber may be dark and may include a mirror. The measuring unit may further include a heating element that may increase the temperature of the reagent and/or water sample if necessary to bring the reaction under observation in the desirable measurement range.

Further, in an embodiment, the handheld wireless device may contain LEDs that may emit light at specific wavelengths. Further, a Color Light-to-Digital Converter with IR Filter may convert information that may be reflected reflection or absorption sensed into a digital signal. Further, additional sensors, such as an ambient light sensor and temperature sensor may provide information to help calculate output.

Further, the computing unit may collect the data generated during the measurements, using various sensors. Further, the data may include color, light intensity, temperature, and external ambient light. Further, the computing unit may generate a multi-variable sequenced raw data set. Further, the computing unit may transmit the data to an external server such as a cloud computer. The final results may then be saved on the external server, along with geolocation and device specific and/or user specific information, and reported or made accessible to create a historical data charts for the tests done at various locations, by various devices, over time.

In some embodiments, the measuring unit may include a standalone console including spectrometer that may test one or many prepared samples of water.

Further, the sampling unit may collect a sample from contaminated water. Further, the sampling unit may include a cartridge to filter substance from contaminated water depending on expected contaminations, and quantitatively transfer the sample to a test reagent. For example, hollow fiber cartridges can be used to concentrate and transfer microbial cells to test vial.

Further, the sample may be mixed with a reagent based on functionalized nanoparticles. The functionalized nanoparticles may include gold, silver or other nanoparticles with similar functional properties. The functionalized nanoparticles may be specific to a reagent being used. Further, the reagent may be used to determine the level of microbial and/or chemical contamination and may be contained in a cuvette for secure measurement.

Further, upon mixing the sample to be measured with the reagent, the measuring unit may start to measure changes in absorption or reflection of light or other attributes with the functionalized nanoparticles and may determine a function that may describe a rate and shape of the attribute change. Further, nanoparticles may aggregate in solution and change color due to a shift in max absorption wavelength as aggregation may change due to Surface Plasmon Resonance (SPR). As the nanoparticles attached to contaminants to be are detected, the degree of aggregation may change and as a result, the max absorption wavelength may change in a measurable way.

The disclosed method may include performing pre-calibration of measuring unit may be performed. The measuring unit may read the baseline set of values for environmental attributes before testing, including but not limited to temperature, lighting conditions without reagent and without vial in place, and pre-calibrates the measuring unit as necessary.

As the ratio of the wavelength changes is independent of outside standards, device calibration to external standards may be redundant. Further, the measuring device may measure changes in sensors (light, temperature, ambient light, room temperature, LED intensity, etc.) at a frequency of several times a second and may collect hundreds of data points over a time of up to minutes or hours depending on the test. Further, adjustments to offset the sensory values are made, if necessary.

Further, the reagent test may be selected and validated. The contaminant to test for may be selected, and the measuring unit may determine if attributes and ratios of the reagent vial inserted without the sample mixed in may be within expected ranges. The ratios may be comprised of sensors readings of visible light with and without the vial inserted.

Further, the water sample to be tested for contamination may be prepared. Further, when contamination or measurement is related to a non-organic contaminant, sampling may directly be done from the contaminated water by a dropper. When the contamination is related to microbial, sampling may be taken by a syringe and passed through a specially designed and constructed concentration filter for related microbial. The filter may concentrate and ready the sample for the transfer of contaminants into the reagent vial.

Further, the prepared sample may be mixed with functionalized nanoparticle reagents. The measuring unit may take continuous readings multiple times a second before and after the sample is mixed with the reagent in a vial. The measuring unit may also simultaneously take readings of environmental attributes. Further, the sample may be considered ready for analysis when at least one of the factors of time or measurement attributes such as the wavelength of light may have changed sufficiently.

Further, the measuring unit may measure the reaction in progress and may take continuous readings of attributes of reaction along with environmental factors including but not limited to temperature and ambient light readings. Depending on the contamination test, the process may transmit data in intervals as required.

Further, the measuring unit may transmit data to the computing unit. The measuring unit may contain software that may transmit the data collected from the sensors, as well as the reaction data collected with the reagent before and after mixing a prepared sample with reagent, including environmental factors in a sequenced manner in a prescribed format. The transmission can occur in a wired or wireless fashion to the related computing unit which may be a smartphone, cloud server, desktop computer or a combination thereof. Further, the data may be consumed by a software application on the computing unit with the likes capable of connecting and receiving data from the measuring unit.

Further, the computing unit may analyze the data received to create an estimated range of probable contamination. The data received from the measuring unit may include pre-calibration, environmental and sample reaction data to determine the baseline and rate of changes in each characteristic. Further, the rate of changes of characteristics may be compared with the baseline data, as well as other relative pre-computed delta ranges, to try to fit with known models or trend-line functions that may include all the relevant variables for specific contaminant testing underway.

Further, coefficients of the multivariable equation produced, along with various statistical analysis data (standard deviation, regression analysis parameters) for various sensors including red, green, blue, ambient light, temperature and light emitted shifts are then used to estimate the concentration and type of contaminant using a built-in, predetermined equation, developed using predictive analysis methods, that generates a contaminant level most probable value and a range of values based on previously collected statistical data. The output may produce a probable range of estimated contamination with an estimated measurement range.

If the output is within a pre-defined detection limit, it may be calculated as the most probable value for the contaminant concentration. Using the Standard Deviation (SD) from trend-line for the data points collected, and the environmental variables, the computing device may also calculate a range of results that may represent an adjusted range of +/−SD (range may include a normal range of values). If the upper or lower limit of the range is outside the detection limit, the value calculated may be a pre-defined detection limit. The computing unit may receive and/or try to geolocate sampling information and make any adjustments to the output to allow for better precision of the probable output range. If the data is insufficient to produce a result with reasonable confidence, the computing unit may request more data from the measuring unit. Once enough data is received and a probable range of contamination can be made, a raw output is created as described above.

Further, the computing unit may stop measuring data and the final output is then sent to the display output. The function for calculating the contaminant levels may be housed on the computing unit, on a mobile application, or in the cloud server, which may receive the data, calculates the response, and returns the data to the computing device. The computing device may then display the output to the user in various formats including on-screen mobile display, PDF report and excel file.

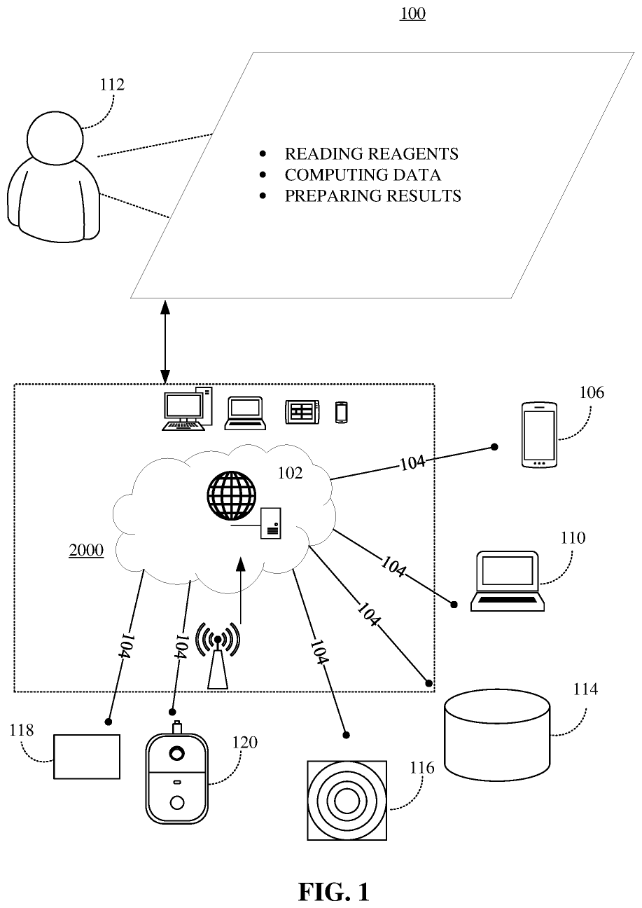

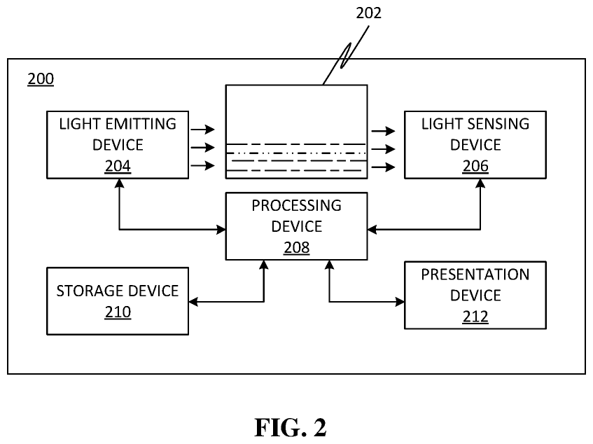

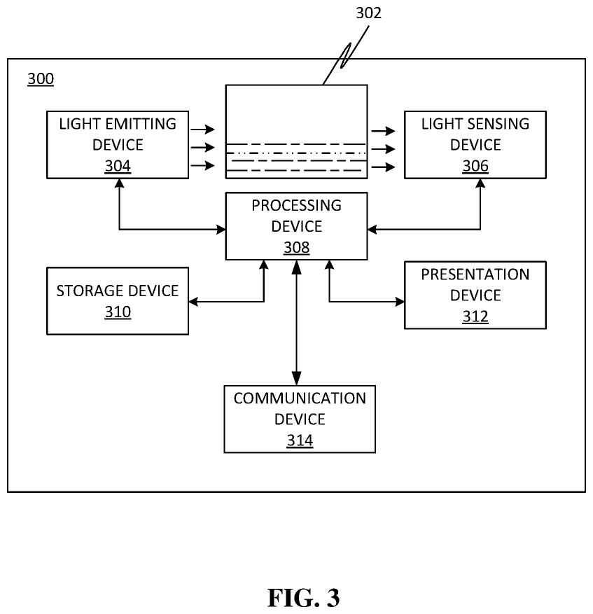

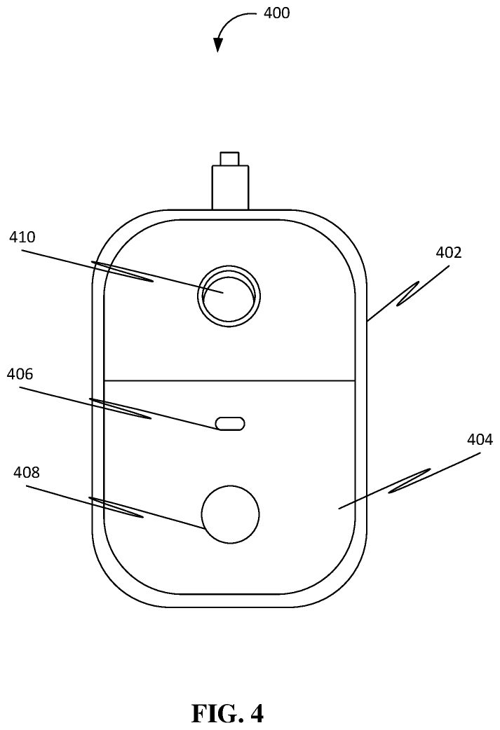

Referring now to figures, FIG. 1 is an illustration of an online platform 100 consistent with various embodiments of the present disclosure. By way of non-limiting example, the online platform 100 to facilitate a system of measuring contamination of a water sample may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, sensors 116, a system 118 (such as a system 200 and a system 300 described in detail in conjunction with FIG. 2 and FIG. 3 below), and a measuring device 120 (such as a measuring device 400 described in detail in conjunction with FIG. 4 below) over a communication network 104, such as, but not limited to, the Internet. Further, users of the online platform 100 may include relevant parties such as, but not limited to, end-users, researchers, government employees and so on. Accordingly, in some instances, electronic devices operated by the one or more relevant parties may be in communication with the platform 100.

A user 112, such as the one or more relevant parties, may access online platform 100 through a web-based software application or browser. The web-based software application may be embodied as, for example, but not be limited to, a website, a web application, a desktop application, and a mobile application compatible with a computing device 2000.

FIG. 2 is a block diagram of a system 200 of measuring contamination of a water sample, in accordance with some embodiments. The system 200 may include a mixture container 202 comprising a mixture chamber, a sample opening (not shown), and a nano reagent opening (not shown). The mixture chamber may be an internal volume of the mixture container 202. Further, each of the sample opening and the nano reagent opening may lead in to the mixing chamber. Further, the mixture chamber is configured for storing a mixture of the water sample and a nano reagent. Further, the water sample may include at least one contaminant. Further, the nano reagent may interact with the at least one contaminant of the water sample. Further, the nano reagent may include a suspension of a plurality of functionalized nanoparticles. Further, the mixture of the nano reagent and the water sample may create a reaction that enables the measurement of the level of microbial load or inorganic contaminants such as Chlorine, Lead or Hydrogen Peroxide.

In some embodiments, the system 200 may facilitate measurement of the at least one contaminant comprising a microbial load in the water sample. Further, the microbial load may include bacteria.

In further embodiments, the system 200 may include a concentration filter (not shown) disposed proximal to the sample opening, wherein the concentration filter is configured for receiving the water sample with a first microbial concentration, wherein the concentration filter is configured for dispensing the water sample with a second microbial concentration, wherein the second microbial concentration is greater than the first microbial concentration. Further, the concentration filter may enable concentration of microbial cells in the water sample for measurement.

In some embodiments, the water sample may be contained in a sampling unit. Further, the sampling unit may be couplable with the sample opening. Further, the sampling unit may include a volumetric sample collection device based on the at least one contaminant. Further, the volumetric sample collection device may include a dropper for the at least one contaminant associated with a first group may include at least one non-organic contaminant. Further, the volumetric sample collection device may include a syringe for the at least one contaminant associated with a second group comprising at least one microbial contaminant (such as the bacteria).

Further, the system 200 may include a light emitting device 204 disposed proximal to the mixing chamber. Further, the light emitting device 204 is configured for emitting an incident light wave corresponding to at least one predetermined attribute. Further, the incident light wave is directed to pass through the mixture. Further, the at least one predetermined attribute may include one or more of wavelength, frequency, and amplitude of the incident light wave.

Further, the system 200 may include a light sensing device 206 disposed proximal to the mixing chamber. Further, the light sensing device 206 is configured for receiving a transmitted light wave. Further, the transmitted light wave is associated with the incident light wave. Further, the transmitted light wave is associated with at least one attribute. Further, the at least one attribute may include one or more of wavelength, frequency, and amplitude of the incident light wave after being passed through the mixture.

Further, the system 200 may include a processing device 208 communicatively coupled with each of the light emitting device 204 and the light sensing device 206. Further, the processing device 208 may be configured for analyzing the at least one predetermined attribute and the at least one attribute. Further, the processing device 208 may be configured for generating a notification based on the analyzing.

Further, the system 200 may include a storage device 210 configured for storing the notification.

Further, the system 200 may include a presentation device 212 communicatively coupled with the processing device 208. Further, the presentation device 212 is configured for presenting the notification.

Further, the system 200 may include a heating element (not shown). Further, the heating element may be configured for increasing temperature of the mixture. Further, increasing the temperature of the mixture may facilitate measurement of the at least one contaminant.

Further, the system 200 may include at least one sensor (such as the sensors 116) communicatively coupled with the processing device 208. Further, each sensor of the at least one sensor is configured for generating a sensor data. Further, the sensor data is associated with the mixture. Further, the processing device 208 is configured for analyzing the sensor data to generate a notification. Further, the presentation device 212 is configured for presenting the notification.

In some embodiments, the at least one sensor may include a termination sensor which may be configured for detecting termination of the measurement of the contaminants in the water sample. Further, the termination sensor may be configured for generating a termination data. Further, the processing device 208 may be configured for analyzing the sensor data to generate an alert.

Further, the light emitting device 204 may be configured for emitting a first incident light wave corresponding to at least one first predetermined attribute. Further, the first incident light wave may be directed to pass through a medium proximal to the light emitting device 204 and the light sensing device 206. For example, the medium may be air. Further, the light sensing device 206 may be configured for receiving a first transmitted light wave. Further, the first transmitted light wave may be associated with the first incident light wave. Further, the first transmitted light wave may be associated with at least one first attribute. Further, the processing device 208 may be configured for analyzing the at least one first predetermined attribute and the at least one first attribute to generate a notification. Further, the presentation device 212 may be configured for presenting the notification. This may be used for performing pre-calibration of the system 200.

In some embodiments, the system 200 may include a communication device (not shown) communicatively coupled with the processing device 208. Further, the communication device may be configured for transmitting the notification to at least one user device (such as the mobile devices 106) associated with a user.

In some embodiments, the processing device 208 may be configured for comparing the notification and a predetermined criterion to generate a score. Further, the predetermined criterion may include a measure of approval associated with the notification. Further, the processing device 208 may be configured for performing one of accepting and rejecting the notification based on the score. The processing device 208 will measure the changes in light emitted when passing through the mixing chamber with sample and nano reagent as well as the sample temperature, with a frequency of at least one reading per second for a period of 1-10 minutes. The readings are then plotted against time and the changes over time are analysed using an algorithm that analyses the type of trendline and the slope and fit of the data to the trendline (the kinetic rate of the reaction). The trendline slope is then compared with pair values for trendline type, slope value and microbial cell concentration at various temperatures from predetermined criterion stored in a database. The algorithm will compare the values obtained, the curve fit (r2) and predict the concentration of microbial cells in the sample tested.

In some embodiments, the nano reagent interacts with the at least one contaminant of the water sample.

FIG. 3 is a block diagram of a system 300 of measuring contamination of a water sample, in accordance with some embodiments. Further, the system 300 may include a mixture container 302 comprising a mixture chamber, a sample opening, and a nano reagent opening. The mixture chamber may be an internal volume of the mixture container 302. Further, each of the sample opening and the nano reagent opening may lead into the mixing chamber. Further, the mixture chamber may be configured for storing a mixture of the water sample and a nano reagent, wherein the water sample comprises at least one contaminant. Further, the nano reagent may include a plurality of functionalized nanoparticles.

In some embodiments, the system 300 may facilitate measurement of the at least one contaminant comprising a microbial load in the water sample. Further, the microbial load may include the bacteria.

In further embodiments, the system 300 may include a concentration filter (not shown) disposed proximal to the sample opening, wherein the concentration filter is configured for receiving the water sample with a first microbial concentration, wherein the concentration filter is configured for dispensing the water sample with a second microbial concentration, wherein the second microbial concentration is greater than the first microbial concentration.

Further, in some embodiments, the system 300 facilitates measurement of the at least one contaminant in the water sample from a chemical contaminant group comprising free chlorine, hydrogen peroxide, and lead.

According to some embodiment, the reagent may interact with the at least one contaminant of the water sample. Further, the reagent may be contained in a cuvette for measurement.

In some embodiments, the water sample may be contained in a sampling unit. Further, the sampling unit is couplable with the sample opening. Further, the sampling unit may include a volumetric sample collection device based on the at least one contaminant. Further, the volumetric sample collection device may include a dropper for the at least one contaminant associated with a first group comprising at least one non-organic contaminant. Further, the volumetric sample collection device may include a syringe for the at least one contaminant associated with a second group comprising at least one microbial contaminant.

Further, the system 300 may include a light emitting device 304 disposed proximal to the mixing chamber. Further, the light emitting device 304 is configured for emitting an incident light wave corresponding to at least one predetermined attribute. Further, the incident light wave is directed to pass through the mixture. Further, the at least one predetermined attribute may include one or more of wavelength, frequency, and amplitude of the incident light wave.

Further, the system 300 may include a light sensing device 306 disposed proximal to the mixing chamber. Further, the light sensing device 306 is configured for receiving a transmitted light wave. Further, the transmitted light wave is associated with the incident light wave. Further, the transmitted light wave is associated with at least one attribute. Further, the at least one attribute may include one or more of wavelength, frequency, and amplitude of the incident light wave after being passed through the mixture.

Further, the system 300 may include a processing device 308 communicatively coupled with each of the light emitting device 304 and the light sensing device 306. Further, the processing device 308 may be configured for analyzing the at least one predetermined attribute and the at least one attribute. Further, the processing device 308 may be configured for generating a notification based on the analyzing.

Further, the system 300 may include a storage device 310 configured for storing the notification.

Further, the system 300 may include a presentation device 312 communicatively coupled with the processing device 308. Further, the presentation device 312 may be configured for presenting the notification.

According to some embodiments, the presentation device 312 may include at least one light emitting diode. Further, the at least one light emitting diode may be configured for indicating termination of the measurement of the at least one contamination.

Further, the system 300 may include a communication device 314 communicatively coupled with the processing device 308. Further, the communication device 314 may be configured for transmitting the notification to at least one user device associated with a user.

According to some embodiments, the processing device 308 may be configured for comparing the notification and a predetermined criterion to generate a score. Further, the predetermined criterion may include a measure of approval associated with the notification. Further, the processing device 308 may be configured for performing one of accepting and rejecting the notification based on the score. The processing device 308 will measure the changes in light emitted when passing through the mixing chamber with sample and nano reagent as well as the sample temperature, with a frequency of at least one reading per second for a period of 1-10 minutes. The readings are then plotted against time and the changes over time are analysed using an algorithm that analyses the type of trendline and the slope and fit of the data to the trendline (the kinetic rate of the reaction). The trendline slope is then compared with pair values for trendline type, slope value and chemical concentration at various temperatures from predetermined criterion stored in a database. The algorithm will compare the values obtained, the curve fit (r2) and predict the concentration of microbial cells in the sample tested.

Further, the system 300 may include at least one sensor (not shown) communicatively coupled with the processing device 308. Further, each sensor of the at least one sensor may be configured for generating a sensor data. Further, the sensor data may be associated with the mixture. Further, the processing device 308 is configured for analyzing the sensor data to generate a notification. Further, the presentation device 312 is configured for presenting the notification.

In some embodiments, the at least one sensor may include a termination sensor which may be configured for detecting termination of the measurement of the contaminants in the water sample. Further, the termination sensor may be configured for generating a termination data. Further, the processing device 308 may be configured for analyzing the sensor data to generate an alert.

In some embodiments, the system 300 may include a heating element (not shown) that may be configured for increasing a temperature of the mixture. Further, the increasing the temperature of the mixture may facilitate measurement of the at least one contaminant.

Further, the light emitting device 304 may be configured for emitting a first incident light wave corresponding to at least one first predetermined attribute. Further, the first incident light wave is directed to pass through a medium proximal to the light emitting device 304 and the light sensing device 306. Further, the medium may be air. Further, the light sensing device 306 may be configured for receiving a first transmitted light wave. Further, the first transmitted light wave may be associated with the first incident light wave. Further, the first transmitted light wave may be associated with at least one first attribute. Further, the processing device 308 may be configured for analyzing the at least one first predetermined attribute and the at least one first attribute to generate a notification. Further, the presentation device 312 may be configured for presenting the notification.

Further, in some embodiments, the reagent interacts with the at least one contaminant of the water sample. Further, the reagent is contained in a cuvette (not shown) for measurement.

FIG. 4 is a top view of a measuring device 400 configured for measuring contamination of a water sample, in accordance with some embodiments. Further, the measuring device (or unit) 400 may be a handheld wireless or wired device/spectrometer that houses a chamber that allows for the measurement of the reaction with minimal interference. This chamber may be dark and may also include a mirror. Further, the measuring device 400 may include a heating element that increases the temperature of the reagent and/or water sample if necessary to bring the reaction under observation in desirable measurement range

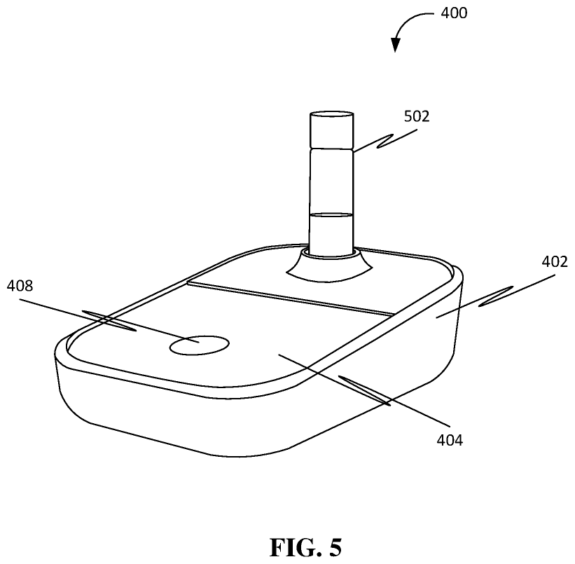

Further, the measuring device 400 may include a silicone cover 402, and a clear lens 404. The clear lens 404 provides a second surface that allows for the flexibility for placement of LED indicators and graphics. Further, the measuring device 400 may include an artwork opening 406 for an ambient light sensor (in the sensors 116). Further, the measuring device 400 may include LED's built into a button 408. Further, the measuring device 400 may include an opening 410 for inserting cuvette or vial with nanoparticles. FIG. 5 shows a top right-side perspective view of the measuring device 400 with a cuvette 502 placed in the opening 410. The opening 410 may have a circular shape, rectangular shape or square shape.

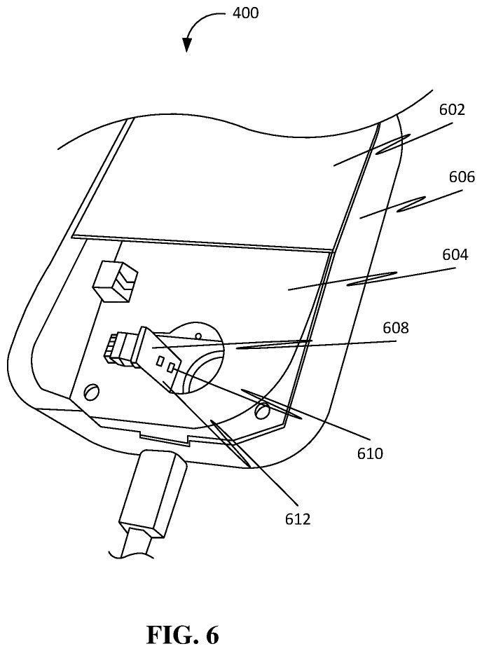

FIG. 6 is a partial top left-side perspective view of internal structure of the measuring device 400, in accordance with some embodiments. The measuring device 400 may include a control pad 602 with embedded ambient sensors. Further, the measuring device 400 may include a printed circuit board 604 with embedded firmware and wireless chips. Further, the measuring device 400 may include a casing 606 that absorbs light. Further, the measuring device 400 may include a circuit board 608 with LEDs and sensors. Further, the measuring device 400 may include LEDs 610 in one or more colors such as green and red. Further, the measuring device 400 may include a light to digital converter 612, such as the Red, Green, Blue, and Clear-light-sensing (RGBC). Further, the measuring device 400 may include a temperature sensor.

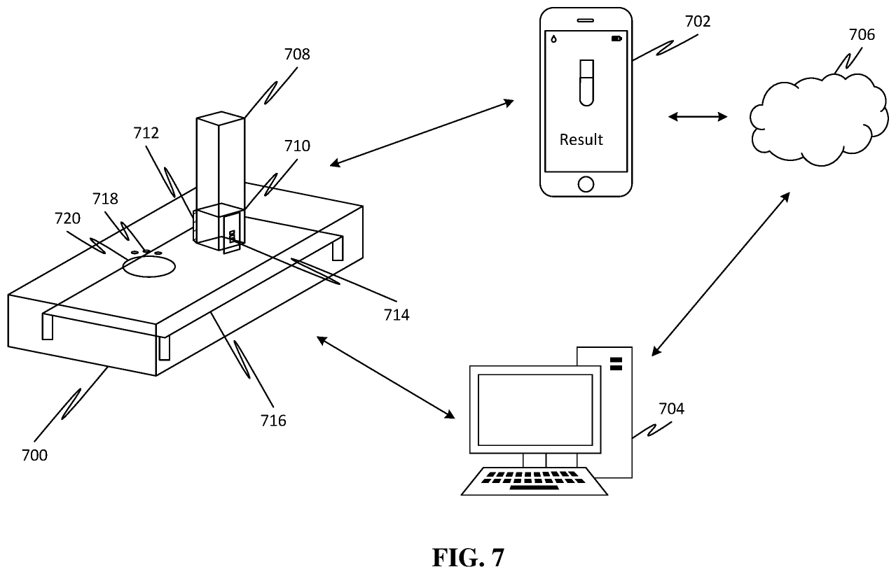

FIG. 7 is a schematic showing communication between a measuring unit 700, a user mobile device 702 (such as the mobile device 106), a user computer 704 (such as the other electronic devices 110) and a cloud server 706 (such as the centralized server 102), in accordance with some embodiments.

Further, the measuring unit 700 may include a cuvette 708, with a water sample mixed with functionalized nanoparticles, placed in an opening 710. The cuvette 708 enters a chamber via the opening 710. This chamber is dark and may include a mirror 712. Further, the chamber may include LEDs 714. Further, the chamber may include RGBC sensors.

Further, the measuring unit 700 may include a PCB 716 with a CPU, a firmware and a wireless chip. Further, the measuring unit 700 may include ambient light sensors 718. Further, the measuring unit 700 may include a power button 720.

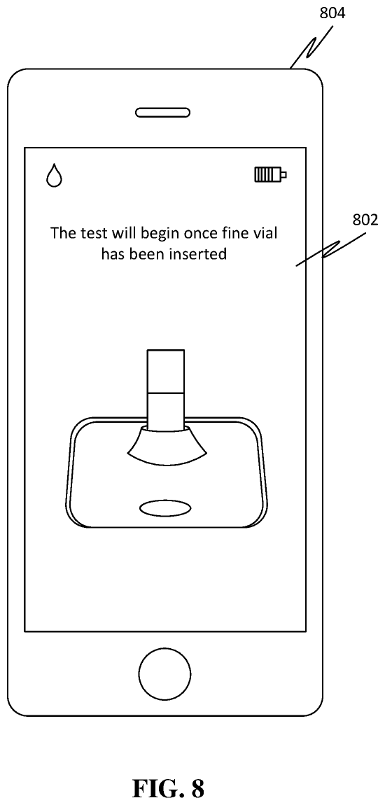

As shown in FIGS. 8, 9, 10, a user may initiate a test, monitor a test and review the results of a test via at least one of the user mobile device 702 and the user computer 704. The test may be conducted by the measuring unit 700.

Further, the test results may be sent to the cloud server 706. Further, the cloud server 706 may be configured for performing data interpretation functions.

FIG. 8 is a snapshot of a user interface 802 of a smartphone application installed on a smartphone 804 (such as the user mobile device 702), in accordance with some embodiments. The user interface 802 may allow a user to initiate a test on the measuring unit 700. Further, the smartphone 804 may communicate with the measuring unit 700 via a wireless or a wired connection.

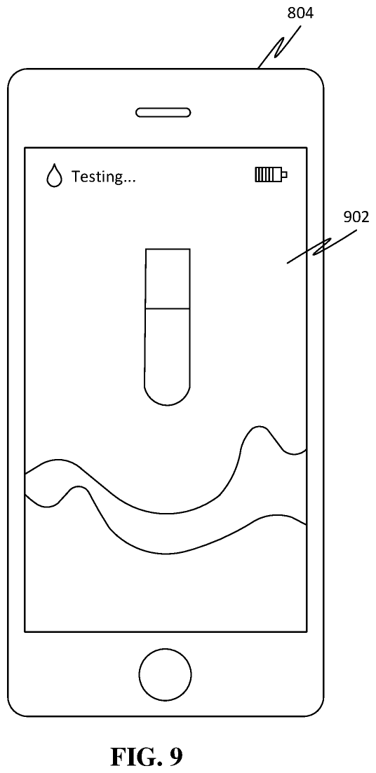

FIG. 9 is a snapshot of a user interface 902 of a smartphone application installed on a smartphone 804, in accordance with some embodiments. The user interface 902 may be shown when a test is under progress.

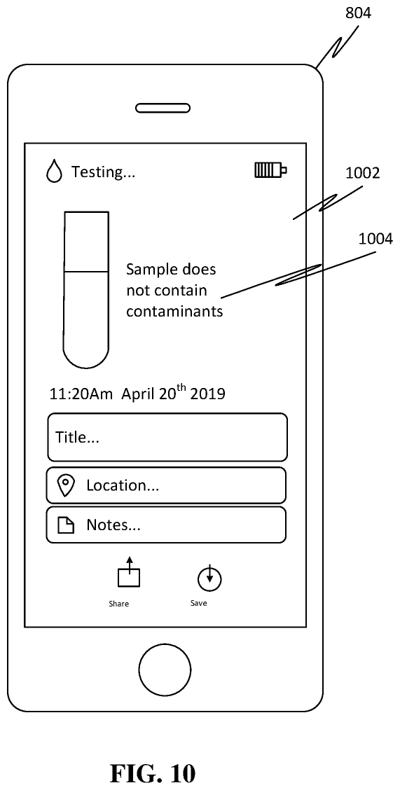

FIG. 10 is a snapshot of a user interface 1002 of a smartphone application installed on a smartphone 804, in accordance with some embodiments. The user interface 1002 may be shown to display the test results. For example, a test result may include a text 1004 that says “Sample does not contain contaminants”.

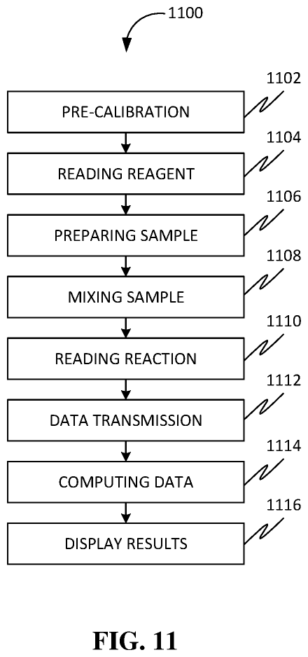

FIG. 11 is a flowchart of a method 1100 of operating a measuring unit (such a measuring unit 700), in accordance with some embodiments. At 1102, the method 1100 may include performing pre-calibration. Further, at 1104, the method 1100 may include reading reagent. Further, at 1106, the method 1100 may include preparing a sample. Further, at 1108, the method 1100 may include mixing the sample. Further, at 1110, the method 1100 may include reading a reaction. Further, at 1112, the method 1100 may include performing data transmission. Further, at 1114, the method 1100 may include computing data. Further, at 1116, the method 1100 may include displaying the results.

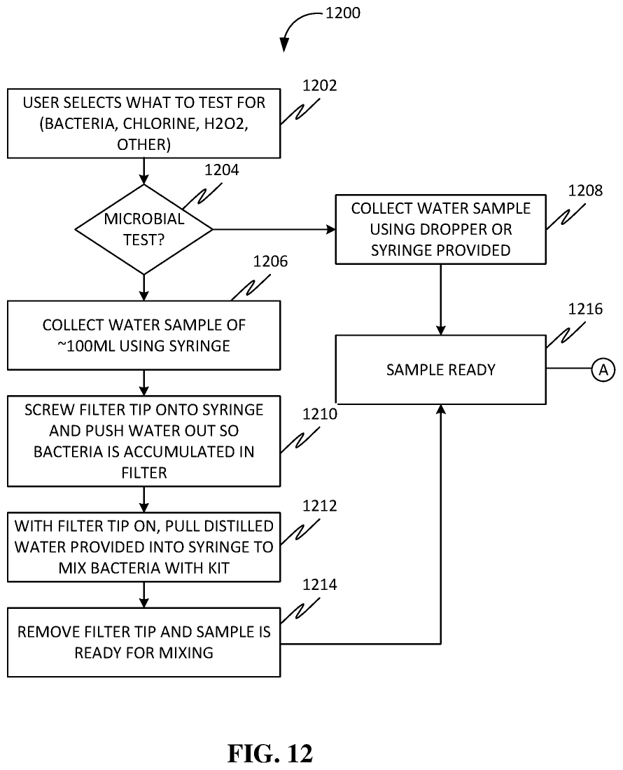

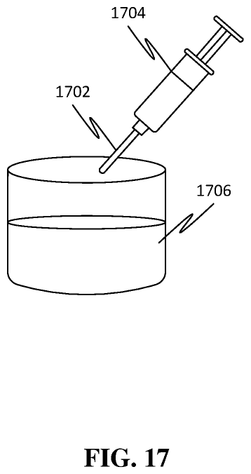

FIG. 12 is a flowchart of a method 1200 of operating a measuring unit (such a measuring unit 700), in accordance with some embodiments. At 1202, the method 1200 may include a user selecting what to test for (such as for microbial load, chlorine, hydrogen peroxide, etc.). Further, at 1204, the method 1200 may include determining if the microbial test has to be performed. If it is determined that the microbial test has to be performed, then at 1206, the method 1200 may include collecting water samples (approx. 100 ml) using a syringe. Further, at 1210, the method 1200 may include screwing a filter tip 1702 onto a syringe 1704 and pushing water out to obtain a water sample 1706, such that the microbial load (or cells) are accumulated in a filter. Further, at 1212, the method 1200 may include pulling distilled water (or other wash agent), with the filter tip on, into the syringe to mix microbial load (or cells) with kit. Further, at 1214, the method 1200 may include removing the filter tip and obtaining the ready sample for mixing. FIG. 17 is a schematic showing the filter tip 1702 screwed onto the syringe 1704 used to prepare the water sample 1706, in accordance with some embodiments.

However, at 1204, if it is determined that the microbial test is not required, then at 1208, the method 1200 may include collecting a water sample using a dropper or a syringe. Therefore, at 1216, the method 1200 may include obtaining the ready sample.

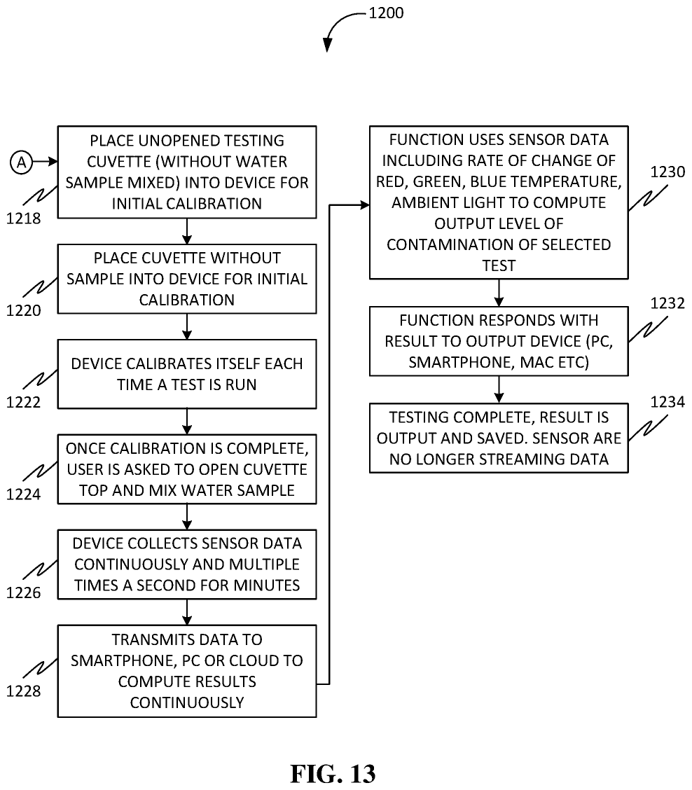

FIG. 13 is a continuation flowchart of the method 1200 shown in FIG. 12. Further, at 1218, the method 1200 may include placing an unopened testing cuvette (without water sample mixed) into a measuring unit for initial calibration. Further, at 1220, the method 1200 may include placing the cuvette without sample into the device for initial calibration. Further, at 1222, the method 1200 may include the measuring unit calibrating itself each time a test is run. Further, at 1224, the method 1200 may include asking a user to open the cuvette top and mix water sample, once calibration is complete. Further, at 1226, the method 1200 may include the measuring unit collecting sensor data continuously and multiple times. Further, at 1228, the method 1200 may include transmitting data to at least one of a smartphone, a personal computer and a cloud server to compute results continuously.

Further, at 1230, the method 1200 may include using sensor data (including rate of change of red, green, blue temperature, ambient light) to compute the output level of contamination of a selected test. Further, at 1232, the method 1200 may include responding with a result to an output device (a personal computer, a smartphone, a mac etc.). Further, at 1234, the method 1200 may include indicating that the test is complete. Further, the result may be output and saved. Further, the streaming of the sensor data may be stopped.

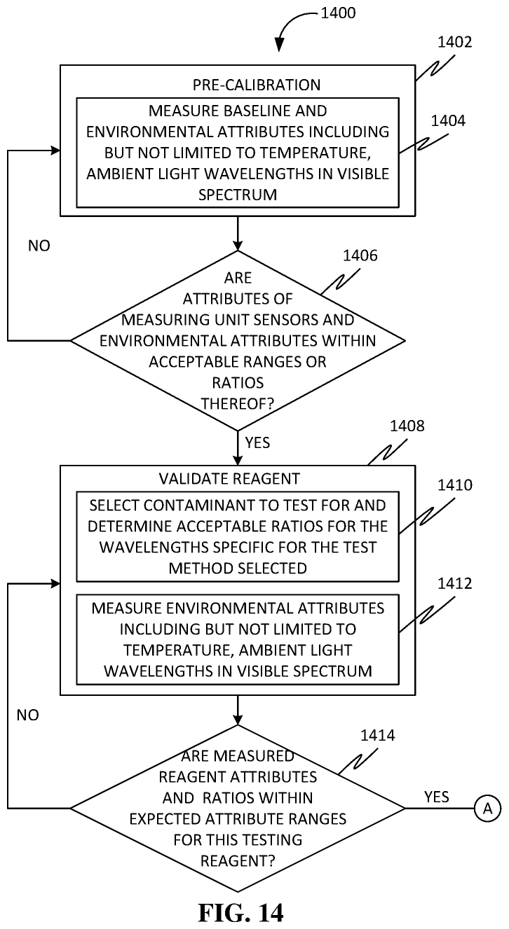

FIG. 14 is a flowchart of a method 1400 of operating a measuring unit (such a measuring unit 700), in accordance with some embodiments. At 1402, the method 1400 may include performing pre-calibration. Further, at 1404, the method 1400 may include measuring baseline and environmental attributes including, but not limited to temperature, ambient light wavelengths in the visible spectrum.

Further, at 1406, the method 1400 may include determining if attributes of measuring unit sensors and environmental attributes are within acceptable ranges or ratios thereof. If it is determined that the attributes of measuring unit sensors and environmental attributes are not within acceptable ranges or ratios thereof, then the method 1400 goes to 1402.

However, at 1406, if it is determined that the attributes of measuring unit sensors and environmental attributes are within acceptable ranges or ratios thereof, then at 1408, the method 1400 may include validating a reagent. Further, at 1410, the method 1400 may include selecting contaminants to test for and determining acceptable ratios for the wavelengths specific for the test method selected. Further, at 1412, the method 1400 may include measuring environmental attributes including, but not limited to temperature, ambient light wavelengths in the visible spectrum.

Further, at 1414, the method 1400 may include determining if measured reagent attributes and ratios are within expected attribute ranges for this testing reagent. If it is determined that the measured reagent attributes and ratios are not within expected attribute ranges for this testing reagent, the method 1400 goes to 1408.

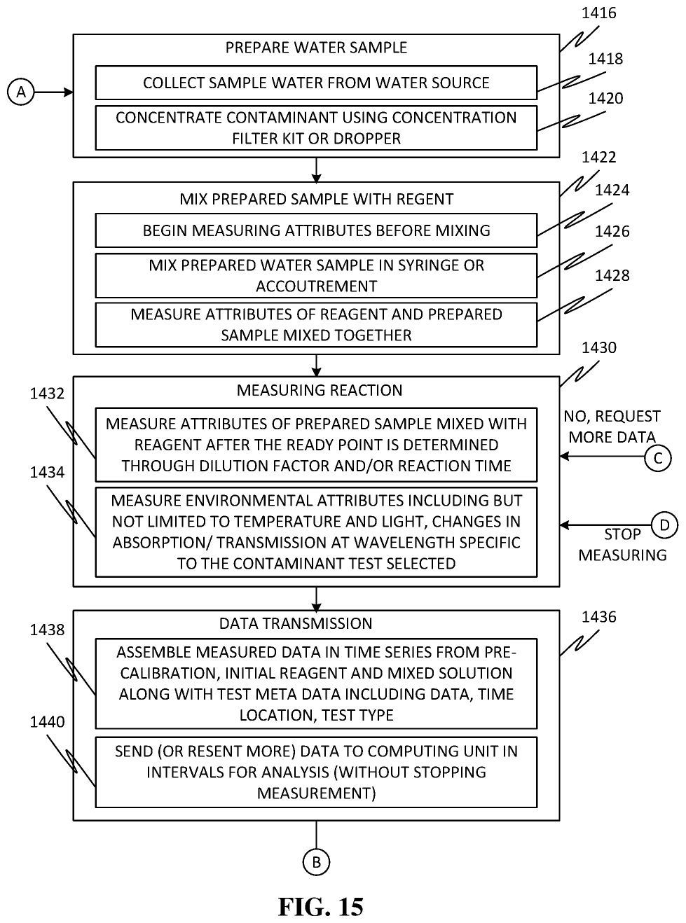

However, at 1414, if it is determined that the measured reagent attributes and ratios are within expected attribute ranges for this testing reagent, then, at 1416, the method 1400 may include preparing a water sample (as shown in FIG. 15). FIG. 15 is a continuation flowchart of the method 1400 shown in FIG. 14.

Further, at 1418, the method 1400 may include collecting the water sample from a water source. Further, at 1420, the method 1400 may include concentrating the contaminant using a concentration filter kit or a dropper.

Further, at 1422, the method 1400 may include mixing the prepared water sample with a reagent. Further, at 1424, the method 1400 may include beginning measuring attributes before mixing. Further, at 1426, the method 1400 may include mixing the prepared water sample in a syringe or an accouterment. Further, at 1428, the method 1400 may include measuring attributes of reagent and prepared water sample mixed together.

Further, at 1430, the method 1400 may include measuring reaction. Further, at 1432, the method 1400 may include measuring attributes of prepared samples mixed with reagent after the ready point is determined through dilution factor and/or reaction time. Further, at 1434, the method 1400 may include measuring environmental attributes including, but not limited to, temperature and light, changes in absorption/transmission at wavelength specific to the contaminant test selected.

Further, at 1436, the method 1400 may include performing data transmission. Further, at 1438, the method 1400 may include assembling measured data in time series from pre-calibration, initial reagent and mixed solution along with test metadata including data, time location, test type. Further, at 1440, the method 1400 may include sending (or resending more) data to a computing unit in intervals for analysis (without stopping measurement).

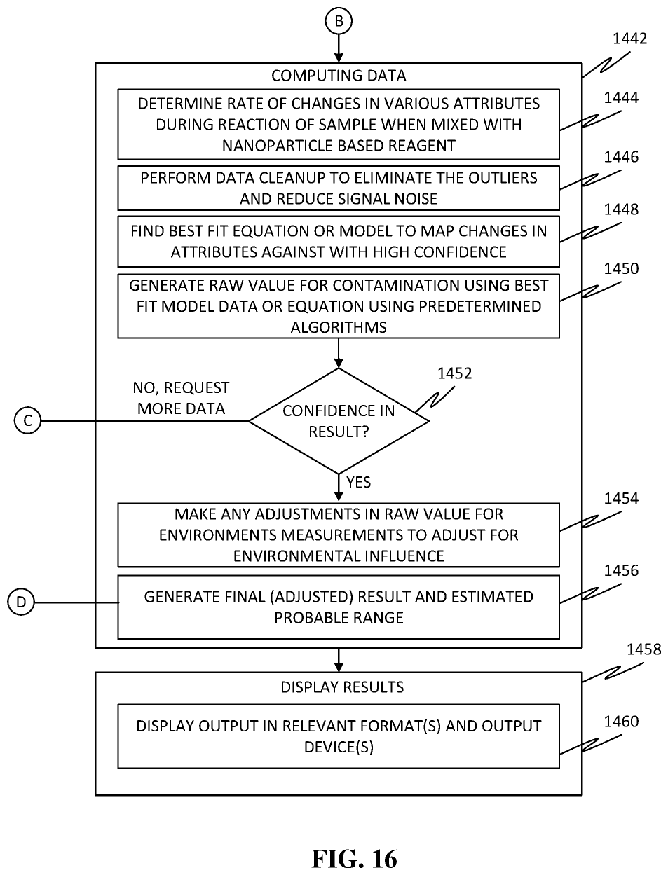

Further, at 1442, the method 1400 may include computing data (as shown in FIG. 16). FIG. 16 is a continuation flowchart of the method 1400 shown in FIG. 14.

Further, at 1444, the method 1400 may include determining the rate of changes in various attributes during the reaction of the sample when mixed with nanoparticle-based reagent. Further, at 1446, the method 1400 may include performing data cleanup to eliminate the outliers and reduce signal noise. Further, at 1448, the method 1400 may include finding the best-fit equation or model to map changes in attributes against with high confidence. Further, at 1450, the method 1400 may include generating raw value for contamination using best fit model data or equation using predetermined algorithms.

Further, at 1452, the method 1400 may include determining confidence level in the result. If it is determined that the confidence level in the result is low, then more data is requested. However, if it is determined that the confidence level in the result is high, then, at 1454, the method 1400 may include making any adjustments in raw value for environment measurements to adjust for environmental influence. Further, at 1456, the method 1400 may include generating a final (adjusted) result and an estimated probable range.

Further, at 1458, the method 1400 may include displaying results. Further, at 1460, the method 1400 may include displaying an output in relevant format(s) and output device(s).

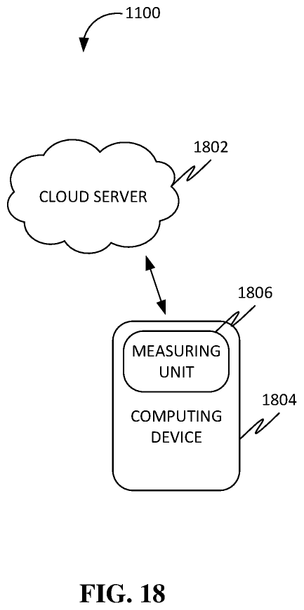

FIG. 18 is a schematic illustrating communication between a cloud server 1802 and a computing device 1804, in accordance with some embodiments. The computing device 1804 may include a measuring unit 1806 (such as the measuring unit 400).

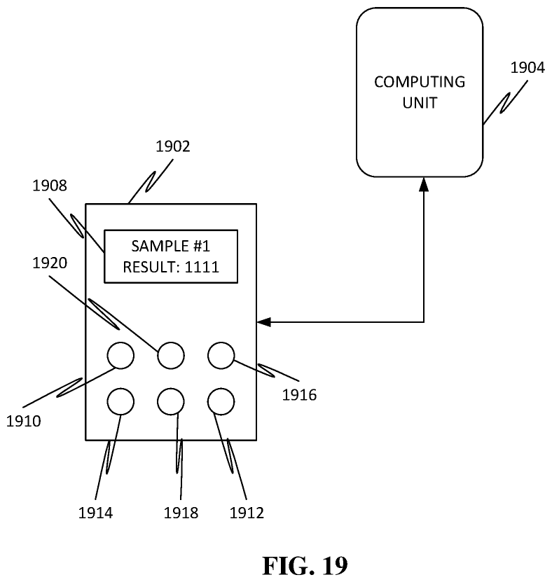

FIG. 19 is a schematic illustrating communication between a measuring unit 1902 and a computing unit 1904, in accordance with some embodiments. Further, the measuring unit 1902 may include multiple openings 1910-1920 to hold multiple sample holders. Further, the measuring unit 1902 may include a display 1908 to display results. Alternatively, the measuring unit 1902 may include an embedded computing device instead of the external computing device 1904.

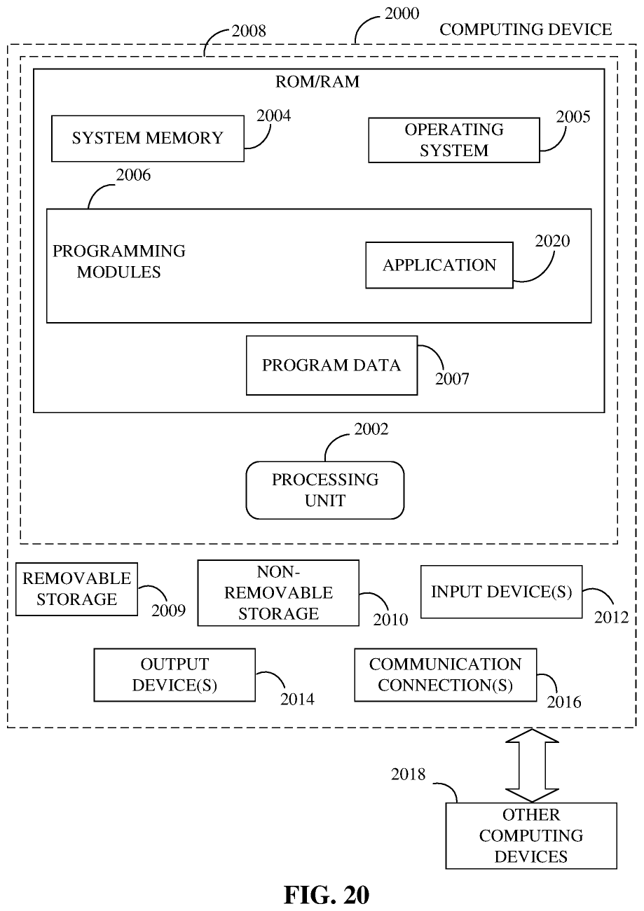

With reference to FIG. 20, a system consistent with an embodiment of the disclosure may include a computing device or cloud service, such as computing device 2000. In a basic configuration, computing device 2000 may include at least one processing unit 2002 and a system memory 2004. Depending on the configuration and type of computing device, system memory 2004 may comprise, but is not limited to, volatile (e.g. random-access memory (RAM)), non-volatile (e.g. read-only memory (ROM)), flash memory, or any combination. System memory 2004 may include operating system 2005, one or more programming modules 2006, and may include a program data 2007. Operating system 2005, for example, may be suitable for controlling computing device 2000's operation. In one embodiment, programming modules 2006 may include image-processing module, machine learning module. Furthermore, embodiments of the disclosure may be practiced in conjunction with a graphics library, other operating systems, or any other application program and is not limited to any particular application or system. This basic configuration is illustrated in FIG. 20 by those components within a dashed line 2008.

Computing device 2000 may have additional features or functionality. For example, computing device 2000 may also include additional data storage devices (removable and/or non-removable) such as, for example, magnetic disks, optical disks, or tape. Such additional storage is illustrated in FIG. 20 by a removable storage 2009 and a non-removable storage 2010. Computer storage media may include volatile and non-volatile, removable and non-removable media implemented in any method or technology for storage of information, such as computer-readable instructions, data structures, program modules, or other data. System memory 2004, removable storage 2009, and non-removable storage 2010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2000. Any such computer storage media may be part of device 2000. Computing device 2000 may also have input device(s) 2012 such as a keyboard, a mouse, a pen, a sound input device, a touch input device, a location sensor, a camera, a biometric sensor, etc. Output device(s) 2014 such as a display, speakers, a printer, etc. may also be included. The aforementioned devices are examples and others may be used.

Computing device 2000 may also contain a communication connection 2016 that may allow device 2000 to communicate with other computing devices 2018, such as over a network in a distributed computing environment, for example, an intranet or the Internet. Communication connection 2016 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 2004, including operating system 2005. While executing on processing unit 2002, programming modules 2006 (e.g., application 2020 such as a media player) may perform processes including, for example, one or more stages of methods, algorithms, systems, applications, servers, databases as described above. The aforementioned process is an example, and processing unit 2002 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may, in fact, be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

The following is claimed:

1. A system of measuring contamination of a water sample, wherein the system comprising:
a mixture container comprising a mixture chamber, a sample opening, and a nano reagent opening, wherein each of the sample opening and the nano reagent opening lead in to the mixing chamber, wherein the mixture chamber is configured for storing a mixture of the water sample and a nano reagent, wherein the water sample comprises at least one contaminant, wherein the nano reagent comprises a plurality of functionalized nanoparticles;
a light emitting device disposed proximal to the mixing chamber, wherein the light emitting device is configured for emitting an incident light wave corresponding to at least one predetermined attribute, wherein the incident light wave is directed to pass through the mixture;
a light sensing device disposed proximal to the mixing chamber, wherein the light sensing device is configured for receiving a transmitted light wave, wherein the transmitted light wave is associated with the incident light wave, wherein the transmitted light wave is associated with at least one attribute;
a processing device communicatively coupled with each of the light emitting device and the light sensing device, wherein the processing device is configured for:
analyzing the at least one predetermined attribute and the at least one attribute; and
generating a notification based on the analyzing:
a storage device configured for storing the notification; and
a presentation device communicatively coupled with the processing device, wherein the presentation device is configured for presenting the notification.

2. The system of claim 1, wherein the system facilitates measurement of the at least one contaminant comprising a microbial load in the water sample.

3. The system of claim 2 further comprising a concentration filter disposed proximal to the sample opening, wherein the concentration filter is configured for receiving the water sample with a first microbial concentration, wherein the concentration filter is configured for dispensing the water sample with a second microbial concentration, wherein the second microbial concentration is greater than the first microbial concentration.

4. The system of claim 1, wherein the system comprises a heating element, wherein the heating element is configured for increasing temperature of the mixture, wherein increasing the temperature of the mixture facilitates measurement of the at least one contaminant.

5. The system of claim 1, wherein the system comprises at least one sensor communicatively coupled with the processing device, wherein each sensor of the at least one sensor is configured for generating a sensor data, wherein the sensor data is associated with the mixture, wherein the processing device is configured for analyzing the sensor data to generate a notification, wherein the presentation device is configured for presenting the notification.

6. The system of claim 1, wherein the light emitting device is configured for emitting a first incident light wave corresponding to at least one first predetermined attribute, wherein the first incident light wave is directed to pass through a medium proximal to the light emitting device and the light sensing device, wherein the light sensing device is configured for receiving a first transmitted light wave, wherein the first transmitted light wave is associated with the first incident light wave, wherein the first transmitted light wave is associated with at least one first attribute, wherein the processing device is configured for analyzing the at least one first predetermined attribute and the at least one first attribute to generate a notification, wherein the presentation device is configured for presenting the notification.

7. The system of claim 1, wherein the system comprises a communication device communicatively coupled with the processing device, wherein the communication device is configured for transmitting the notification to at least one user device associated with a user.

8. The system of claim 1, wherein the processing device is configured for comparing the notification and a predetermined criterion to generate a score, wherein the predetermined criterion comprises a measure of approval associated with the notification.

9. The system of claim 8, wherein the processing device is configured for performing one of accepting and rejecting the notification based on the score.

10. The system of claim 1, wherein the nano reagent interacts with the at least one contaminant of the water sample.

11. A system of measuring contamination of a water sample, wherein the system comprising:
a mixture container comprising a mixture chamber, a sample opening, and a nano reagent opening, wherein each of the sample opening and the nano reagent opening lead into the mixing chamber, wherein the mixture chamber is configured for storing a mixture of the water sample and a nano reagent, wherein the water sample comprises at least one contaminant, wherein the nano reagent comprises a plurality of functionalized nanoparticles;
a light emitting device disposed proximal to the mixing chamber, wherein the light emitting device is configured for emitting an incident light wave corresponding to at least one predetermined attribute, wherein the incident light wave is directed to pass through the mixture;
a light sensing device disposed proximal to the mixing chamber, wherein the light sensing device is configured for receiving a transmitted light wave, wherein the transmitted light wave is associated with the incident light wave, wherein the transmitted light wave is associated with at least one attribute;
a processing device communicatively coupled with each of the light emitting device and the light sensing device, wherein the processing device is configured for:
analyzing the at least one predetermined attribute and the at least one attribute; and
generating a notification based on the analyzing:
a storage device configured for storing the notification;
a presentation device communicatively coupled with the processing device, wherein the presentation device is configured for presenting the notification; and
a communication device communicatively coupled with the processing device, wherein the communication device is configured for transmitting the notification to at least one user device associated with a user.

12. The system of claim 11, wherein the system facilitates measurement of the at least one contaminant comprising a microbial load in the water sample.

13. The system of claim 12 further comprising a concentration filter disposed proximal to the sample opening, wherein the concentration filter is configured for receiving the water sample with a first microbial concentration, wherein the concentration filter is configured for dispensing the water sample with a second microbial concentration, wherein the second microbial concentration is greater than the first microbial concentration.

14. The system of claim 11, wherein the system facilitates measurement of the at least one contaminant in the water sample from a chemical contaminant group comprising free chlorine, hydrogen peroxide, and lead.

15. The system of claim 11, wherein the system comprises at least one sensor communicatively coupled with the processing device, wherein each sensor of the at least one sensor is configured for generating a sensor data, wherein the sensor data is associated with the mixture, wherein the processing device is configured for analyzing the sensor data to generate a notification, wherein the presentation device is configured for presenting the notification.

16. The system of claim 11, wherein the presentation device comprises at least one light emitting diode, wherein the at least one light emitting diode is configured for indicating termination of the measurement of the at least one contamination.

17. The system of claim 11, wherein the light emitting device is configured for emitting a first incident light wave corresponding to at least one first predetermined attribute, wherein the first incident light wave is directed to pass through a medium proximal to the light emitting device and the light sensing device, wherein the light sensing device is configured for receiving a first transmitted light wave, wherein the first transmitted light wave is associated with the first incident light wave, wherein the first transmitted light wave is associated with at least one first attribute, wherein the processing device is configured for analyzing the at least one first predetermined attribute and the at least one first attribute to generate a notification, wherein the presentation device is configured for presenting the notification.

18. The system of claim 11, wherein the processing device is configured for comparing the notification and a predetermined criterion to generate a score, wherein the predetermined criterion comprises a measure of approval associated with the notification.

19. The system of claim 18, wherein the processing device is configured for performing one of accepting and rejecting the notification based on the score.

20. The system of claim 11, wherein the nano reagent interacts with the at least one contaminant of the water sample.
